(19) United States
(12) Patent Application Publication
(10) Pub. No.: US 2017/0072184 A1
(43) Pub. Date: Mar. 16, 2017

(54) WEARABLE SMART DRUG-DELIVERY SYSTEM

(71) Applicant: XIAMEN MICROTECH BIOSCIENCES CO., LTD., Xiamen (CN)

(72) Inventors: Joseph Zhili Huang, Xiamen (CN); Jason Jinglong CHEN, Xiamen (CN)

(73) Assignee: XIAMEN MICROTECH BIOSCIENCES CO., LTD, Xiamen (FJ)

(21) Appl. No.: 15/312,182

(22) PCT Filed: Oct. 22, 2015
(86) PCT No.: PCT/CN2015/092546

§ 371 Date: Nov. 18, 2016

(30) Foreign Application Priority Data
Oct. 31, 2014 (CN) 201410600426.0

(51) Int. Cl. A61N 1/04; A61M 37/00; A61N 1/30

(52) U.S. Cl. CPC A61N 1/0448; A61N 1/303; A61M 37/00; A61M 2037/0007

(57) ABSTRACT

Provided is a wearable smart drug-delivery system, including a power supply, a drug sac structure, a repository, and an electrode structure. The wearable smart drug-delivery system can accurately and precisely control the dosage and delivery time of an ionized pharmaceutical agent, reducing and preventing uncontrolled doses of transdermally delivered drugs. A plurality of drugs may be stored in one repository or a plurality of repositories in the wearable smart drug-delivery system, and may, at the same time or at different times, be delivered into the body by means of one or a plurality of power supplies; furthermore, storing the plurality of drugs in the plurality of repositories can prevent reactions and interferences between drugs.

### BACKGROUND OF THE INVENTION

1. Field of the Invention

The present invention relates to a wearable smart drug-delivery system.

2. Description of the Prior Art

Drugs provide therapeutic effects in different diseases. A patient must receive the medical treatment at a certain time or a certain time mode of administration, or drugs must be maintained at a certain concentration level to achieve the best therapeutic effect. Unfortunately, patients sometimes do not take the medicine at the required interval of time or at the right time. Besides, the high acidity of the stomach or the first-pass effect of the liver may impact oral drugs to result in a failure of part or all of the oral drugs.

To overcome these problems, medicament may be administered through the skin. Common parenteral administration is by injecting different doses of the drug with a syringe or continuous infusion. These methods may not be appropriate for the patients who need a long-term treatment because the repeated injury of needles and an intravenous infusion device will limit the freedom of movement for patients and give the patients pain.

A more comfortable drug delivery device is administered through the epidermis. In the field of Chinese medicine, transdermal patches or plasters are widely used. Administration in the form of a patch is generally performed over a period of time (e.g., one day to one week) to provide continuous doses, without the patient's active request for administration. Routine applications of drugs through the skin are: nicotine, hormones and some analgesics (such as fentanyl) and other dosage form changed drugs, such as donepezil hydrochloride, aspirin, ibuprofen, Ritalin. Its applications include nicotine patches for smoking cessation; patches for contraceptive use by continuously releasing low levels of estrogen and progestin to the skin. This epidermal delivery device is portable, comfortable and very suitable for patients with fear of needles. The main advantages of epidermal administration are: sustained doses at designed release rates; avoidance of gastrointestinal and liver first-pass effects on oral drugs; no patient's intervention in administration, but patients may administer or stop the medicine at any time. However, its advantages for some of the drugs are its shortcomings: the maintenance of a sustained dose of the body, leading to the body or disease resistance and drug resistance; the body's physiological function or pathological phenomenon is a clear circadian rhythm, administration should be based on drug bioavailability, plasma concentration, the change of the circadian rhythm during the process of metabolism and excretion. A constant dose will affect the efficacy of treatment and produce adverse reactions and side effects.

Iontophoresis is a physical process of ion current diffusion driven by an external electric field medium, so that soluble ionic salt drugs can enter the body under the action of current so as to achieve therapeutic purposes. Iontophoresis devices were first started in 1900. In 1934, British patent No. 410,009 described an iontophoretic device which includes a plurality of electrodes, a material containing a therapeutic ingredient or medicament through transdermal delivery, and a battery to supply the power energy for iontophoretic delivery. The device is accepted because it reduces the influence of the administration on the daily life of the patient, and is the realization of the early electric ion penetration administration. Recently, some U.S. patents published the related techniques for iontophoretic therapy, such as U.S. Pat. No. 7,844,327 to Anderson et al., U.S. Pat. No. 7,945,320 to Durand, U.S. Pat. No. 8,386,030 to Kananmura et., U.S. Pat. No. 8,463,373 to Anderson, U.S. Pat. No. 8,666,486 to Dombdisclose, and the like, which disclose iontophoretic devices and a number of applications. This means people are interested in this method for drug delivery.

In general, in an iontophoretic device, at least two electrodes are used to get contact with the skin directly, and a transdermal accelerator is required when in use. This causes a great damage to the skin. An electrode is called an active electrode and acts on chemicals, compounds, medicaments, or drugs to be delivered into the body. The other is called a counter electrode. The two electrodes together with the skin and the power supply form a closed voltage or current loop. For example, if a drug ion is positive (positive charge), the positive electrode (anode) is a closed loop between the active electrode and the counter electrode (cathode). If the drug ion is negative (negative charge), the negative electrode is the active electrode and the positive electrode is the counter electrode. The most prominent advantage of the iontophoresis is the control of drug delivery and time.

### SUMMARY OF THE INVENTION

The primary objective of the present invention is to provide a wearable smart drug-delivery system which is convenient for use.

According to an aspect of the present invention, a wearable smart drug-delivery system comprises a drug sac structure and a first power supply. The drug sac structure includes a first repository and a second repository therein. The second repository is annular. The first repository is disposed in the second repository. The first repository is provided with a first electrode therein. One side of the first repository has a first opening. The first repository is filled with a gelatinous drug. The second repository is provided with a second electrode therein. One side of the second repository has a second opening. The second repository is filled with a gelatinous electrolyte. Positive and negative electrodes of the first power supply are connected with the first electrode and the second electrode, respectively.

Preferably, the first opening is opposite to the first electrode, and the second opening is opposite to the second electrode.

Preferably, the first opening is provided with a first transdermal film, and the second opening is provided with a second transdermal film.

Preferably, the first repository is spaced apart from the second repository by more than 10 mm.

Preferably, the gelatinous drug is detachably disposed in the first repository, and the gelatinous electrolyte is detachably disposed in the second repository.

Preferably, the wearable smart drug-delivery system further comprises at least one third repository. The third repository is disposed in the first repository. The third repository is provided with a third electrode therein. One side of the third repository has a third opening. The third opening is provided with a third transdermal film. The third repository is filled with a gelatinous drug. The first power supply is connected with the third electrode.

Preferably, the third opening is opposite to the third electrode.

According to another aspect of the present invention, a wearable smart drug-delivery system comprises a drug sac structure and a first power supply. The drug sac structure includes a first repository, a second repository, and a central repository therein. The second repository is annular. The first repository is annular and located between the second repository and the central repository. The first repository is provided with a first electrode therein. One side of the first repository has a first opening. The first repository is filled with a gelatinous drug. The second repository is provided with a second electrode therein. One side of the second repository has a second opening. The second repository is filled with a gelatinous electrolyte. The central repository is provided with a central electrode therein. One side of the central repository has a central opening. The central repository is filled with the gelatinous electrolyte. Positive and negative electrodes of the first power supply are connected with the first electrode and between the second electrode and the central electrode, respectively.

Preferably, the first opening is provided with a first transdermal film, the second opening is provided with a second transdermal film, and the central opening is provided with a central transdermal film.

Preferably, the first repository is divided into at least one third repository. The third repository is provided with a third electrode therein. One side of the third repository has a third opening. The third opening is provided with a third transdermal film. The third repository is filled with a gelatinous drug.

The present invention has the following advantages. The dosage of delivery time of the ionized drug can be accurately and precisely controlled. The dosage of the drug in contact with the skin through the skin is far lower than the dosage of iontophoretic permeation through the skin so as to minimize or avoid the uncontrolled dosage of the drug through transdermal delivery. A plurality of drugs can be stored in one repository or a plurality of repositories of the present invention. By means of one or more power supplies, the drugs can be delivered into the body at the same time or at different times. Furthermore, storing the plurality of drugs in the plurality of repositories can prevent reactions and interferences between drugs. There is no need to use a transdermal accelerator, reducing skin irritation.

### BRIEF DESCRIPTION OF THE DRAWINGS

FIG. 1 is a perspective view in accordance with a first embodiment of the present invention;

FIG. 2 is a sectional view in accordance with the first embodiment of the present invention;

FIG. 3 is a perspective view in accordance with a second embodiment of the present invention; and

FIG. 4 is a perspective view in accordance with a third embodiment of the present invention.

### DETAILED DESCRIPTION OF THE PREFERRED EMBODIMENTS

The present invention discloses a wearable smart drug-delivery system. The wearable smart drug-delivery system comprises a drug sac structure and a first power supply. The drug sac structure includes a first repository and a second repository therein. The second repository is annular. The first repository is disposed in the annular second repository. The first repository is provided with a first electrode therein. One side of the first repository has a first opening. The first opening is provided with a first transdermal film. The first repository is filled with a gelatinous drug. The second repository is provided with a second electrode therein. One side of the second repository has a second opening. The second opening is provided with a second transdermal film. The second repository is filled with a gelatinous electrolyte. The positive and negative electrodes of the first power supply are connected with the first electrode and the second electrode, respectively. The first opening is opposite to the first electrode, and the second opening is opposite to the second electrode. The first repository is spaced apart from the second repository by more than 10 mm. The gelatinous drug is detachably disposed in the first repository. The gelatinous electrolyte is detachably disposed in the second repository.

The present invention further comprises at least one third repository. The third repository is disposed in the first repository. The third repository is provided with a third electrode therein. One side of the third repository has a third opening. The third opening is provided with a third transdermal film. The third repository is filled with a gelatinous drug. The first power supply is connected with the third electrode. The third opening is opposite to the third electrode.

In another embodiment, the drug sac structure of the present invention comprises a first repository, a second repository, and a central repository. The second repository is annular. The first repository is also annular and located between the second repository and the central repository. The first repository is provided with a first electrode therein. One side of the first repository has a first opening. The first opening is provided with a first transdermal film. The first repository is filled with a gelatinous drug. The second repository is provided with a second electrode therein. One side of the second repository has a second opening. The second opening is provided with a second transdermal film. The second repository is filled with a gelatinous electrolyte. The central repository is provided with a central electrode therein. One side of the central repository has a central opening. The central opening is provided with a central transdermal film. The central repository is filled with a gelatinous electrolyte. The positive and negative electrodes of the first power supply are connected with the first electrode and between the second electrode and the central electrode, respectively.

In the embodiment of the present invention, through the transdermal film the drug is ionized to be delivered to the skin through the above-mentioned transdermal film, and it may be called a permeable film which is a thin film structure having uniform pores with a certain bore diameter.

Embodiments of the present invention will now be described, by way of example only, with reference to the accompanying drawings.

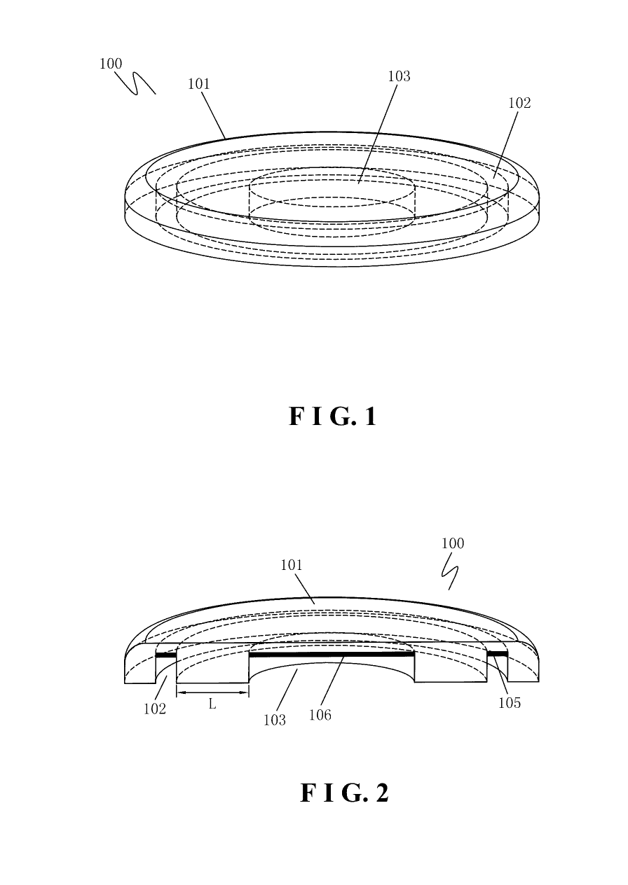

FIG. 1 and FIG. 2 show a first embodiment of the present invention. A wearable smart drug-delivery system 100 of the present invention comprises a circular drug sac structure 101 and a first power supply (not shown in the drawings). The drug sac structure 101 includes a circular first repository 103 and an annular second repository 102. The first repository 103 is provided with a first electrode 106 therein. The second repository 102 is provided with a second electrode 105 therein. As shown in FIG. 2, the first electrode 106 and the second electrode 105 are installed on the top walls of the respective repositories. The bottom of the first repository 103 is provided with a first transdermal film (not shown in the drawings). The bottom of the second repository 102 is provided with a second transdermal film (not shown in the drawings). The first power supply is applied to the first electrode in the first repository and the second electrode in the second repository.

A part of the drug sac structure 101 may be made of a synthetic material or silicone rubber. The size of the wearable smart drug-delivery system 100 of the present invention is 75 mm in diameter and 6 mm in height, but not limited thereto. Other sizes may be applicable. The first repository 103 has a diameter of 20 mm and a height of 3 mm. The second repository 102 may have an inner diameter of 40 mm, an outer diameter of 45 mm, and a height of 3 mm. Other sizes may be applicable. The bottom areas of the second repository 102 and the first repository 103 are the same as soon as possible. The distance L between the second repository 102 and the first repository 103 is greater than 10 mm to ensure that the current can be uniformly dispersed to the skin.

For a positive ionized drug, the gelatinous ionized dug may be stored in the first depository 103. The gel in the second repository 102 may be a gel only containing electrolytic ions. The positive terminal of the first power supply is connected with the first electrode 106 in the first repository 103, and the negative terminal of the first power supply is connected with the second electrode 105 in the second repository 102. When the first power supply is actuated, the positive ionized drug in the first repository 103 is delivered from the first repository 103 through the first transdermal film to the skin beneath the first transdermal film under the action of coulombs and transmitted through the skin into the body. Meanwhile, by controlling the intensity of the output current or voltage of the first power supply, the ionized drug can be delivered into the body in a dose.

The positive and negative ionized drugs may be mixed with a gelatinous substance to form a gelatinous drug to be stored in the first repository 103 or the second repository 102, respectively. The reference gelatinous substrate may be an organogel or a hydrogel. In addition, the hydroxyethyl cellulose may be mixed with the gelatinous substance, but not limited thereto. Other gelatinous substance may be used.

In the design of the drug sac structure 101, the positions corresponding to the first repository 102 and the second repository 103 may be hollow cavity structures. The gelatinous electrolyte and the gelatinous drug can be formed corresponding in size and in shape to the hollow cavity structures of the first repository 102 and the second repository 103, so that the gelatinous electrolyte and the gelatinous drug can be placed therein. In this way, the gelatinous electrolyte and the gelatinous drug can be replaced, and the drug sac structure 101 can be integrated with the wearable smart drug-delivery system 100.

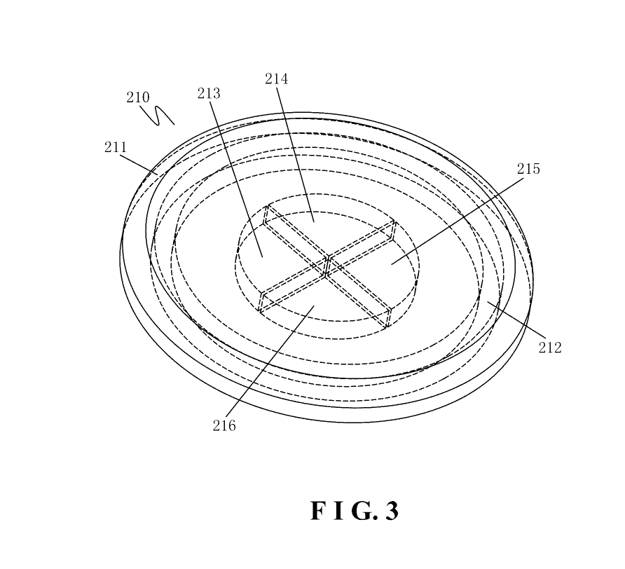

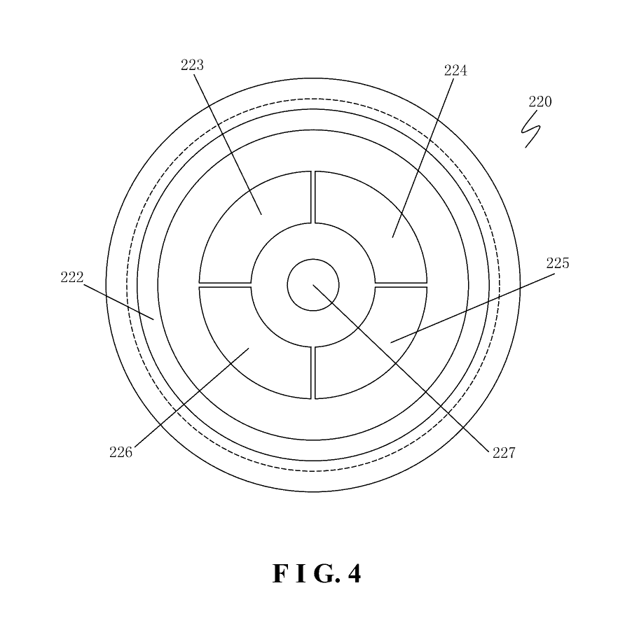

FIG. 3 shows a second embodiment of the present invention. A wearable smart drug-delivery system 210 of the present invention comprises a drug sac structure 211, a first power supply (not shown in the drawing), a first repository 213, a third repository 214, a fourth repository 215, a fifth repository 216, and an annular second repository 212. The top of the first repository 213 is provided with a first electrode (not shown in the drawing). The bottom of the first repository 213 has a first opening (not shown in the drawing). The first opening is provided with a first transdermal film (not shown in the drawing). The top of the third repository 214 is provided with a third electrode (not shown in the drawing). The bottom of the third repository 214 has a third opening (not shown in the drawing). The third opening is provided with a third transdermal film (not shown in the drawing). The top of the fourth repository 215 is provided with a fourth electrode (not shown in the drawing). The bottom of the fourth repository 215 has a fourth opening (not shown in the drawing). The fourth opening is provided with a fourth transdermal film (not shown in the drawing). The top of the fifth repository 216 is provided with a fifth electrode (not shown in the drawing). The bottom of the fifth repository 216 has a fifth opening (not shown in the drawing). The fifth opening is provided with a fifth transdermal film (not shown in the drawing). The top of the second repository 212 is provided with a second electrode (not shown in the drawing). The bottom of the second repository 212 has a second opening (not shown in the drawing). The second opening is provided with a second transdermal film (not shown in the drawing). The wearable smart drug-delivery system 210 of the present invention can be used for four different drugs. The different drugs are placed into the first repository 213, the third repository 214, the fourth repository 215, and the fifth repository 216, respectively. The first power supply is connected with the electrodes of the first repository 213, the third repository 214, the fourth repository 215, and the fifth repository 216 and the electrode of the second repository 212 to achieve the transdermal drug delivery of four different drugs at the same time, alternatively, a plurality of power supplies are respectively connected with the electrodes of the first repository 213 and the second repository 212, the electrodes of the third repository 214 and the second repository 212, the electrodes of the fourth repository 215 and the second repository 212, and the electrodes of the fifth repository 216 and the second repository 212 to achieve the transdermal drug delivery of four different drugs at different times.

FIG. 4 shows a third embodiment of the present invention. A wearable smart drug-delivery system 220 of the present invention comprises a first power supply (not shown in the drawing), a first repository 223, a third repository 224, a fourth repository 225, a fifth repository 226, an annular second repository 222, and a central repository 227. The top of the first repository 223 is provided with a first electrode (not shown in the drawing). The bottom of the first repository 223 has a first opening (not shown in the drawing). The first opening is provided with a first transdermal film (not shown in the drawing). The top of the third repository 224 is provided with a third electrode (not shown in the drawing). The bottom of the third repository 224 has a third opening (not shown in the drawing). The third opening is provided with a third transdermal film (not shown in the drawing). The top of the fourth repository 225 is provided with a fourth electrode (not shown in the drawing). The bottom of the fourth repository 225 has a fourth opening (not shown in the drawing). The fourth opening is provided with a fourth transdermal film (not shown in the drawing). The top of the fifth repository 226 is provided with a fifth electrode (not shown in the drawing). The bottom of the fifth repository 226 has a fifth opening (not shown in the drawing). The fifth opening is provided with a fifth transdermal film (not shown in the drawing). The top of the second repository 222 is provided with a second electrode (not shown in the drawing). The bottom of the second repository 222 has a second opening (not shown in the drawing). The second opening is provided with a second transdermal film (not shown in the drawing). For the current to be distributed more evenly to the area of the wearable smart drug-delivery system, the wearable smart drug-delivery system 220 further comprises the central repository 227. The top of the central repository 227 is provided with a central electrode (not shown in the drawing). The bottom of the central repository 227 has a sixth opening (not shown in the drawing). The sixth opening is provided with a sixth transdermal film (not shown in the drawing). In this embodiment, four different drugs are placed into the first repository 223, the third repository 224, the fourth repository 225, and the fifth repository 226, respectively.

The second electrode of the second repository 222 is connected with the central electrode of the central repository 227. The first power supply is connected between the electrodes of the first repository 223, the third repository 224, the fourth repository 225, and the fifth repository 226 and the electrodes of the second repository 222 and the central repository 227 to achieve the transdermal drug delivery of four different drugs at the same time, alternatively, a plurality of power supplies are respectively connected between the electrode of the first repository 223 and the electrodes of the second repository 222 and the central repository 227; the electrode of the third repository 224 and the electrodes of the second repository 222 and the central repository 227; the electrode of the fourth repository 225 and the electrodes of the second repository 222 and the central repository 227; the electrode of the fifth repository 226 and the electrodes of the second repository 222 and the central repository 227 to achieve the transdermal drug delivery of four different drugs at different times.

Furthermore, the third embodiment of the present invention may further comprise a second power supply (not shown in the drawing), a third power supply (not shown in the drawing), and a fourth power supply (not shown in the drawing). That is, the first power supply is connected between the first electrode and the second electrode as well as the central electrode; namely, between the electrode of the first repository 223 and the electrodes of the second repository 222 and the central repository 227. The second power supply is connected between the third electrode and the second electrode as well as the central electrode. The third power supply is connected between the fourth electrode and the second electrode as well as the central electrode. The fourth power supply is connected between the fifth electrode and the second electrode as well as the central electrode.

The wearable smart drug-delivery system 220 of the present invention can be used for the transdermal drug delivery of four different drugs. Different drugs are placed into the first repository 223, the third repository 224, the fourth repository 225, and the fifth repository 226, respectively. The first power supply is connected between the electrodes of the first repository 223, the third repository 224, the fourth repository 225, and the fifth repository 226 and the electrodes of the second repository 222 and the central repository 227 to achieve the transdermal drug delivery of four different drugs at the same time, alternatively, a plurality of power supplies are respectively connected between the electrode of the first repository 223 and the electrodes of the second repository 222 and the central repository 227; the electrode of the third repository 224 and the electrodes of the second repository 222 and the central repository 227; the electrode of the fourth repository 225 and the electrodes of the second repository 222 and the central repository 227; and the electrode of the fifth repository 226 and the electrodes of the second repository 222 and the central repository 227. Through the first power supply, the second power supply, the third power supply, and the fourth power supply, the wearable smart drug-delivery system 220 is able to achieve the transdermal drug delivery of four different drugs at different times.

The present invention has the following advantages. The dosage of delivery time of the ionized drug can be accurately and precisely controlled. The dosage of the drug in contact with the skin through the skin is far lower than the dosage of iontophoretic permeation through the skin so as to minimize or avoid the uncontrolled dosage of the drug through transdermal delivery. A plurality of drugs can be stored in one repository or a plurality of repositories of the present invention. By means of one or more power supplies, the drugs can be delivered into the body at the same time or at different times. Furthermore, storing the plurality of drugs in the plurality of repositories can prevent reactions and interferences between drugs. There is no need to use a transdermal accelerator, reducing skin irritation.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A wearable smart drug-delivery system, comprising a drug sac structure and a first power supply, the drug sac structure including a first repository and a second repository therein, the second repository being annular, the first repository being disposed in the second repository, the first repository being provided with a first electrode therein, one side of the first repository having a first opening, the first repository being filled with a gelatinous drug, the second repository being provided with a second electrode therein, one side of the second repository having a second opening, the second repository being filled with a gelatinous electrolyte, positive and negative electrodes of the first power supply being connected with the first electrode and the second electrode, respectively.

2. The wearable smart drug-delivery system as claimed in claim 1, wherein the first opening is opposite to the first electrode, and the second opening is opposite to the second electrode.

3. The wearable smart drug-delivery system as claimed in claim 1, wherein the first opening is provided with a first transdermal film, and the second opening is provided with a second transdermal film.

4. The wearable smart drug-delivery system as claimed in claim 1, wherein the first repository is spaced apart from the second repository by more than 10 mm.

5. The wearable smart drug-delivery system as claimed in claim 1, wherein the gelatinous drug is detachably disposed in the first repository, and the gelatinous electrolyte is detachably disposed in the second repository.

6. The wearable smart drug-delivery system as claimed in claim 1, further comprising at least one third repository, the third repository being disposed in the first repository, the third repository being provided with a third electrode therein, one side of the third repository having a third opening, the third opening being provided with a third transdermal film, the third repository being filled with a gelatinous drug, the first power supply being connected with the third electrode.

7. The wearable smart drug-delivery system as claimed in claim 6, wherein the third opening is opposite to the third electrode.

8. A wearable smart drug-delivery system, comprising a drug sac structure and a first power supply, the drug sac structure including a first repository, a second repository, and a central repository therein, the second repository being annular, the first repository being annular and located between the second repository and the central repository, the first repository being provided with a first electrode therein, one side of the first repository having a first opening, the first repository being filled with a gelatinous drug, the second repository being provided with a second electrode therein, one side of the second repository having a second opening, the second repository being filled with a gelatinous electrolyte, the central repository being provided with a central electrode therein, one side of the central repository having a central opening, the central repository being filled with the gelatinous electrolyte, positive and negative electrodes of the first power supply being connected with the first electrode and between the second electrode and the central electrode, respectively.

9. The wearable smart drug-delivery system as claimed in claim 8, wherein the first opening is provided with a first transdermal film, the second opening is provided with a second transdermal film, and the central opening is provided with a central transdermal film.

10. The wearable smart drug-delivery system as claimed in claim 8, wherein the first repository is divided into at least one third repository, the third repository is provided with a third electrode therein, one side of the third repository has a third opening, the third opening is provided with a third transdermal film, the third repository is filled with a gelatinous drug.
